(19) United States
(12) Patent Application Publication
(10) Pub. No.: US 2003/0148377 A1
(43) Pub. Date: Aug. 7, 2003

(54) Binding compounds and methods for identifying binding compounds

(76) Inventors: Kiyotaka Nishikawa, Tokyo (JP); Hung-Sen Lai, Winchester, MA (US); Zhou Songyang, Houston, TX (US); Michael B. Yaffe, Jamaica Plain, MA (US); Lewis C. Cantley, Cambridge, MA (US)

Correspondence Address: WOLF GREENFIELD & SACKS, PC, FEDERAL RESERVE PLAZA, 600 ATLANTIC AVENUE, BOSTON, MA, 02210-2211, US

(21) Appl. No.: 10/017,672

(22) Filed: Dec. 14, 2001

Related U.S. Application Data
(60) Provisional application No. 60/255,586, filed on Dec. 14, 2000.

(51) Int. Cl. G01N 33/53; C12Q 1/48

(52) U.S. Cl. 435/7.1; 435/15

(57) ABSTRACT

The invention provides methods for rapidly determining kinase binding site motifs using a oriented degenerate peptide library approach. The methods involve the selection of peptides by binding affinity. Inhibitors of kinases that include or compete for the binding site motifs determined using the methods also are provided, as are methods and compositions for using these binding site motifs and inhibitors.

### RELATED APPLICATIONS

This application claims the benefit under 35 U.S.C. § 119(e) of U.S. provisional application 60/255,586, filed Dec. 14, 2000, the entire disclosure of which is incorporated herein by reference.

### GOVERNMENT SUPPORT

This work was funded in part by the National Institutes of Health under grant number GM56203. The government may have certain rights in this invention.

### BACKGROUND OF THE INVENTION

Inhibitors of lymphocyte activation are of potential use for treatment of a variety of human diseases, including transplant rejection, autoimmune disease, and inflammation. Activation of T cells requires a subset of protein Tyr kinases that are relatively restricted to these cells, including ZAP-70, ITK, and Lck. The relative importance of these kinases for lymphocyte activation has been evaluated by gene disruption in the mouse. However, the possibility of compensatory expression of other genes during development can complicate the interpretation of gene disruption studies. The availability of specific inhibitors of individual protein Tyr kinases would help clarify the role that these enzymes play in signaling pathways involved in particular cellular responses and reveal the consequence of acute inhibition.

Improved techniques for delivery of peptides to the cell interior have made it feasible to introduce peptides as inhibitors of intracellular signaling pathways. However, to date little progress has been made in identifying high-affinity and high-specificity peptide inhibitors of protein Tyr kinases. Previously, we developed a peptide library technique for identifying optimal substrates of protein kinases that involved determining the consensus sequence of a selected set of phosphopeptide products of the kinase of interest (Songyang et al., 1994, 1995, 1996; U.S. Pat. No. 5,532,167; U.S. Pat. No. 6,004,757; PCT publication WO98/54577; Nishikawa et al., 1997). This technique selects for peptides that are phosphorylated (favoring a high Vₘₐₓ/Kₘ ratio; Nishikawa et al., 1997) which do not necessarily have the highest binding affinity at the catalytic site.

Thus there is a need for the identification of peptides that are not necessarily optimal kinase substrates, but which are optimized for binding affinity to kinases, and thus are useful as, and for the design of, inhibitors of kinases.

### SUMMARY OF THE INVENTION

We have determined consensus optimal kinase binding motifs using a novel affinity-based peptide library screen. Peptides based on this motif inhibited kinase activity with high affinity and high selectivity. A membrane-permeant version of this peptide specifically blocked signaling downstream of the kinase. Our studies extend the range of experimental approaches for probing kinase function in vivo and introduce a technique for developing protein kinase inhibitors that could be useful for a wide range of protein kinases.

According to one aspect of the invention, methods for determining an amino acid sequence binding motif for a phosphorylation site of a kinase are provided. The methods include:

a) contacting the kinase with a peptide library, wherein each peptide comprises a single non-degenerate phosphorylatable amino acid in a fixed position of the peptide and wherein each peptide comprises one or more degenerate amino acids, under conditions which allow for binding of a peptide by the kinase at the phosphorylation site of the kinase;

b) allowing the kinase to bind peptides of the peptide library having a binding site for the kinase phosphorylation site to form kinase-peptide complexes;

c) isolating the kinase-peptide complexes from the unbound peptides;

d) releasing the peptides from the kinase-peptide complexes;

e) isolating the peptides previously bound to the kinase;

f) determining the amino acid sequences of the peptides; and

g) determining an amino acid sequence motif for a binding site of the kinase based upon the relative abundance of different amino acid residues at each degenerate position within the peptides.

In certain embodiments, the peptide library comprises peptides comprising the formula:

(Xaa)ₙ-Zaa-(Xaa)ₘ (SEQ ID NO: 13)

wherein Zaa is the single non-degenerate phosphorylatable amino acid and is selected from the group consisting of Tyr, Ser and Thr,

wherein Xaa is any amino acid except Zaa, and

wherein n and m are integers from 1-10 inclusive.

Preferably, the peptide library is a soluble synthetic peptide library.

In some embodiments, the single non-degenerate phosphorylatable amino acid is tyrosine. In such embodiments, the peptides preferably include the amino acid sequence Xaaₙ-Tyr-Xaaₘ (SEQ ID NO: 14), wherein Xaa is any amino acid except Tyr and wherein n and m are integers from 1-10 inclusive. More preferably, the peptides include the amino acid sequence Xaaₙ-Tyr-Xaaₘ (SEQ ID NO: 15), wherein Xaa is any amino acid except Tyr or Cys and wherein n and m are integers from 1-10 inclusive. Still more preferably, the peptides include the amino acid sequence Xaaₙ-Tyr-Xaaₘ (SEQ ID NO: 16), wherein Xaa is any amino acid except Tyr, Cys or Trp and wherein n and m are integers from 1-10 inclusive. In particularly preferred embodiments, the peptides include the amino acid sequence Xaa₄-Tyr-Xaa₄ (SEQ ID NO: 17), wherein Xaa is any amino acid except Tyr, Cys or Trp.

In other embodiments, the single non-degenerate phosphorylatable amino acid is serine. In such embodiments, the peptides preferably include the amino acid sequence Xaaₙ-Ser-Xaaₘ (SEQ ID NO: 18), wherein Xaa is any amino acid except Ser and wherein n and m are integers from 1-10 inclusive. More preferably, the peptides include the amino acid sequence Xaaₙ-Ser-Xaaₘ (SEQ ID NO: 19), wherein Xaa is any amino acid except Ser or Cys and wherein n and m are integers from 1-10 inclusive. Still more preferably, the peptides comprise the amino acid sequence Xaaₙ-Ser-Xaaₘ (SEQ ID NO: 20), wherein Xaa is any amino acid except Ser, Cys or Trp and wherein n and m are integers from 1-10 inclusive.

In yet other embodiments, the single non-degenerate phosphorylatable amino acid is threonine. In such embodiments, the peptides preferably include the amino acid sequence Xaaₙ-Thr-Xaaₘ (SEQ ID NO: 21), wherein Xaa is any amino acid except Thr and wherein n and m are integers from 1-10 inclusive. More preferably, the peptides include the amino acid sequence Xaaₙ-Thr-Xaaₘ (SEQ ID NO: 22), wherein Xaa is any amino acid except Thr or Cys and wherein n and m are integers from 1-10 inclusive. Still more preferably, the peptides include the amino acid sequence Xaaₙ-Thr-Xaaₘ (SEQ ID NO: 23), wherein Xaa is any amino acid except Thr, Cys or Trp and wherein n and m are integers from 1-10 inclusive.

In other preferred embodiments, the peptide library is contacted with the kinase by application of the library to a substrate to which the kinase is immobilized. In further preferred embodiments, the kinase-peptide complexes are isolated by washing the kinase-peptide complexes in a buffer that permits binding of peptides to the phosphorylation site of the kinase, and/or the peptides are eluted from the kinase-peptide complexes by incubating the kinase-peptide complexes with an elution solution. For these methods, it is preferred that the elution solution has an acidic pH.

According to another aspect of the invention, kinase binding molecules are provided. The molecules include a binding motif for a phosphorylation site of a kinase identified using any of the foregoing methods. In certain embodiments, the kinase binding molecules include a molecule that mediates transport across a plasma membrane. Preferably, the molecule that mediates transport across a plasma membrane is selected from the group consisting of penetratin, Tat, VP22, Pep-1 and fragments thereof that mediate transport across a plasma membrane. Also provided in accordance with this aspect of the invention are compositions include the foregoing kinase binding molecules and a pharmaceutically acceptable carrier, and the use of the kinase binding molecules in the preparation of a medicament.

According to still another aspect of the invention, methods for inhibiting phosphorylation of proteins by a kinase are provided. The methods include contacting the kinase with an amount of the foregoing kinase binding molecules or compositions effective to inhibit the phosphorylation. Also provided are methods for treating a condition that includes phosphorylation of proteins by a kinase. These methods include administering to a subject an amount of the foregoing kinase binding molecules or compositions effective to inhibit the phosphorylation of the proteins by the kinase. Preferably the subject is a mammal, particularly a human.

According to a further aspect of the invention, kinase inhibitors are provided. The kinase inhibitors include a binding motif for a phosphorylation site of a kinase identified according to the foregoing methods, wherein the single non-degenerate phosphorylatable amino acid is replaced by an amino acid that cannot be phosphorylated by the kinase to which the inhibitor binds. In some embodiments, the binding motif comprises SEQ ID NO: 2, or SEQ ID NO: 4. Preferably the kinase inhibitor comprises SEQ ID NO: 4 or SEQ ID NO: 9. In preferred embodiments, the amino acid that cannot be phosphorylated is Tyr and the Tyr is replaced by Phe or a halogenated Phe. In other preferred embodiments, the kinase inhibitors also include a molecule that mediates transport across a plasma membrane. Preferably the molecule that mediates transport across a plasma membrane is selected from the group consisting of penetratin, Tat, VP22, Pep-1 and fragments thereof that mediate transport across a plasma membrane. Also provided in accordance with this aspect of the invention are compositions that include the foregoing kinase inhibitors and a pharmaceutically acceptable carrier, as well as the use of the kinase inhibitors the preparation of a medicament.

The invention in another aspect provides methods for inhibiting phosphorylation of proteins by a kinase. The methods include contacting the kinase with an amount of the foregoing kinase inhibitors or the compositions containing them effective to inhibit the phosphorylation. Also provided are methods for treating a condition that includes phosphorylation of proteins by a kinase. These methods include administering to a subject an amount of the foregoing kinase inhibitor or the compositions containing them effective to inhibit the phosphorylation of the proteins by the kinase. Preferably the subject is a mammal, particularly a human.

Also included as another aspect of the invention are methods for validating a kinase as a target for inhibition for the treatment of a condition. The methods include providing a molecule comprising a binding motif for a phosphorylation site of a kinase as determined by the foregoing methods, contacting a biological sample containing a kinase suspected of being involved in the causation of the condition with the molecule for a time sufficient to permit binding of the molecule and the kinase, and determining the effect of the molecule on one or more biological processes mediated by the kinase. In some embodiments, the biological sample is a cell or an organism. Preferably the cell or organism is a model system for the condition.

In another aspect of the invention, methods for inhibiting a ZAP-70 kinase are provided. The methods include contacting the ZAP-70 kinase with an amount of the foregoing kinase inhibitors, effective to inhibit the ZAP-70 kinase. Preferably the kinase inhibitor includes SEQ ID NO: 4 or SEQ ID NO: 9. Also provided are methods for treating a condition mediated by a ZAP-70 kinase. The methods include administering to a subject in need of such treatment an amount of the foregoing kinase inhibitors or the composition containing them, effective to inhibit the ZAP-70 kinase. In some embodiments, the kinase inhibitor includes SEQ ID NO: 4 or SEQ ID NO: 9. Preferably the subject is a mammal, particularly a human.

According to a further aspect of the invention, methods for inhibiting transcription mediated by a ZAP-70-responsive promoter sequence are provided. The methods include contacting a biological sample, cell or organism that includes a ZAP-70-responsive promoter sequence operably linked to a nucleic acid molecule with an amount of the foregoing kinase inhibitors effective to inhibit the transcription of the nucleic acid molecule mediated by the ZAP-70-responsive promoter sequence. In preferred embodiments, the kinase inhibitor comprises SEQ ID NO: 4 or SEQ ID NO: 9. In other embodiments, the ZAP-70-responsive promoter sequence is an interleukin-2 promoter. Also provided are methods for treating a condition mediated by transcription mediated by a ZAP-70-responsive promoter sequence. The methods include administering to a subject in need of such treatment an amount of the foregoing kinase inhibitors or a composition containing them, effective to inhibit the transcription mediated by the ZAP-70-responsive promoter sequence. In preferred embodiments, the kinase inhibitor comprises SEQ ID NO: 4 or SEQ ID NO: 9. In other embodiments, the ZAP-70-responsive promoter sequence is an interleukin-2 promoter. Preferably the subject is a mammal, particularly a human.

Another aspect of the invention provides methods for identifying a kinase inhibitor compound. The methods include providing a kinase, a kinase inhibitor that binds the kinase, and a candidate kinase inhibitor compound, contacting the kinase with the candidate kinase inhibitor compound and the kinase inhibitor under conditions that permit binding of the kinase inhibitor to the kinase, wherein either or both of the candidate kinase inhibitor compound and the kinase inhibitor are detectable, and wherein either or both of the candidate kinase inhibitor compound and the kinase inhibitor comprises a sequence determined according to the methods described above, separating the kinase from the unbound kinase inhibitor and unbound candidate kinase inhibitor compound, and detecting the amounts of detectable kinase inhibitor and/or the detectable candidate kinase inhibitor compound bound to the kinase as a measure of the presence of a candidate kinase inhibitor compound that competes with the kinase inhibitor for binding to the kinase.

In some embodiments, the methods also include testing the activity of the kinase in the presence of the candidate kinase inhibitor compound, wherein a greater reduction in kinase activity in the presence of the candidate kinase inhibitor compound than in the absence of the candidate kinase inhibitor compound indicates that the candidate kinase inhibitor compound is a kinase inhibitor. In preferred embodiments, the candidate kinase inhibitor compound or the kinase inhibitor comprises a binding motif sequence comprising SEQ ID NO: 13. In other preferred embodiments, the candidate kinase inhibitor compound is a small organic molecule.

Also provided are kinase inhibitor compounds identified according to these methods, and the use of such kinase inhibitor compounds in the preparation of a medicament.

These and other objects and features of the invention are described in greater detail below.

### BRIEF DESCRIPTION OF THE DRAWINGS

FIG. 1 shows that the ZAP-70 binding peptide is a specific and potent competitive inhibitor of ZAP-70 Tyr kinase activity.

FIG. 1A: A glutathione-S-transferase chimera of ZAP-70 was purified from Sf9 cells and assayed for its ability to phosphorylate tubulin in the presence of 0, 50, or 100 μM of peptide 1 (SEQ ID NO: 5) or peptide 2 (SEQ ID NO: 6). The assay was carried out in kinase buffer (5 mM MnCl₂, 5 mM MgCl₂, 25 mM Tris [pH 7.4]), 50 μM ATP, and μCi [γ-³²P]ATP for 5 min at 30° C. ³²P-phosphorylated tubulin was separated on SDS-PAGE, visualized by autoradiography (inset), and quantitated using a molecular imager (Bio-Rad).

FIG. 1B: ZAP-70 was also assayed for its ability to phosphorylate peptide 1 (10 μM) in the presence of 0, 10, or 50 μM of peptide 4 (SEQ ID NO: 9) or peptide 5 (SEQ ID NO: 10). Peptide phosphorylation was determined by spotting the reaction mixture on phosphocellulose and washing away the ³²P-ATP with a phosphate buffer. The polylysine tail of peptide 1 facilitates retention on the phosphocellulose (Songyang et al., 1996). The data are presented as the percentage of the activity present in the absence of inhibitory peptides.

FIG. 1C: Various concentrations of peptide 1 (25-500 μM) were phosphorylated by ZAP-70 in the presence of 0 μM (open circle), 2.5 μM (closed circle), and 5 μM (closed square) of peptide 4. The assay were performed as in FIG. 1B and graphed as a Lineweaver-Burke plot. The best fit to the data indicate competitive inhibition with a K_{I} of 2.1 μM. The data are representative of three experiments.

FIG. 1D: The ZAP-70 Tyr-kinase assay was as described above, using tubulin (5 μM) as substrate. Peptide 4 (closed circles), peptide 5 (open triangles), or peptide 6 (open circles) was added at the indicated concentrations. Peptide 6 is defined in the inset as KLILYLLLL (SEQ ID NO: 4).

FIG. 1E: A glutathione-S-transferase chimera of Lck was purified from Sf9 cells and assayed for the ability to phosphorylate myelin basic protein (5 μM) in the presence or absence of the same three peptides as used in FIG. 1D. The assay was carried out for 5 min at 30° C.

FIG. 1F: Myc-tagged Syk kinase was expressed and purified from COS-7 cells. GST-band III (Kₘ concentration; 0.7 μM for ZAP-70 and 3.4 μM for Syk) was phosphorylated by ZAP-70 (closed circle) or myc-tagged Syk (open circle) in the presence of the indicated amount of peptide 4 for 10 min at 30° C. Phosphorylated tubulin, myelin basic protein, and GST-band III were separated on SDS-PAGE, visualized by autoradiography and quantitated as described in the legend for FIG. 1B. Error bars represent the standard error from 3-4 independent experiments.

FIG. 2 shows that a membrane-permeant form of the ZAP-70 inhibitory peptide inhibits ZAP-70 in intact lymphocytes. Penetratin-containing peptides (FIG. 2A; peptide 7 (SEQ ID NO: 11) or peptide 8 (SEQ ID NO: 12)) were dissolved in DMSO and diluted to 20 μM in 500 μl of serum-free RPMI1640 medium. Ten million Jurkat cells, suspended in 500 μl of serum-free RPMI1640, were added to the peptide solutions for diluted DMSO control and incubated for 30 min at 37° C. Peptide-pretreated cells were treated with 1 μg of the anti-CD3ε mAb at 4° C. for 10 min, followed by another 10 min incubation in the presence of 5 μg of rabbit anti-mouse Ig. Cells were activated by shifting the temperature to 37° C. for 2 min and subsequently lysed in 1% NP40 lysis buffer. Lysates were immunoprecipitated with either the 4G10 anti-phosphotyrosine mAb (FIG. 2B; lanes 1-5), or with anti-PLCγ1 antibody (FIG. 2B; lanes 6-10), or with anti-LAT antibody (FIG. 2D; lane 11-15), and immunoblotted with the HRP-conjugated anti-phosphotyrosine Ab, RC20H. Lanes 1, 2, 6, 7, 11 and 12 corresponded to untreated Jurkat cells; lysates in lanes 3, 8, and 13 were prepared from cells treated with DMSO; lysates in lanes 4, 9, and 14 were prepared from cells treated with peptide 7; lysates in lanes 5, 10, and 15 were prepared from cells treated with peptide 8. (−) indicates nonstimulated cells; (+) indicates stimulation by CD3ε cross-linking. Each blot was stripped and reprobed with anti-PLCγ1 antibody (FIG. 2C) or anti-LAT antibody (FIG. 2E). Jurkat cells were electroporated in the presence of 10 μg of plasmid carrier DNA, 100 ng of Renilla luciferase control vector, and 2.5 μg of a luciferase reporter gene. Transfectants were treated with 10 μM of peptide 7, peptide 8, or DMSO as described above. Treated cells were stimulated with plate-bound anti-CD3ε mAb and 10 ng/ml of PMA (phorbol 12-myristate-13-acetate; FIG. 2F), or 10 ng/ml of PMA plus 2 μM of ionomycin (FIG. 2G) at 37° C. for 2 hr. Luciferase activities from lysates were measured using a Dual-Luciferase Reporter System and Monolight 2010 luminometer.

### DETAILED DESCRIPTION OF THE INVENTION

The invention relates to methods for determining binding motifs for kinases, peptides that contain such motifs, inhibitors containing at least a portion of such motifs and methods for using these compounds.

Recognition of the optimal binding peptide by a kinase depends in part on complementarity between the kinase active site and the sequence surrounding the phosphorylatable amino acid in the binding peptide. Determination of kinase binding site motifs has several important applications. Analogs of optimized binding peptides tailor-made to the kinase provide potent inhibitors useful as lead compounds in drug discovery and further as tools in exploring the biological function of the kinase.

In addition to the ZAP-70 kinase used herein, the invention is generally applicable in identifying inhibitors for any kinase. Thus the methods of the invention can be applied to determine binding motifs of, for example, medically interesting kinases including other Src-family kinases, kinases involved in T-cell and B-cell growth and differentiation, kinases involved in signal transduction, kinases involved in cancer, kinases involved in immune system related diseases, kinases involved in inflammation, kinases involved in heart disease and vascular disease (e.g., atherosclerosis), kinases involved in diabetes, etc. Other kinases of interest will be known to one of skill in the art.

Thus the invention pertains generally to methods for determining binding motifs of kinases, peptides identified by such methods, and inhibitors including such peptides. The method of identifying kinase binding peptides includes contacting a kinase with a degenerate library of peptides, for a sufficient time for binding between the peptides and the kinase. The kinase molecules can be immobilized to facilitate purification of kinase, recovery of the kinase/bound peptide complexes and/or removal of unbound peptides. Kinases can be immobilized by any method known to one of ordinary skill in the art; as shown herein, for example, a kinase can be fused to glutathione-S-transferase to facilitate recovery of the kinase, packing of a kinase/peptide binding column, elution of unbound peptides.

The methods provided herein have the advantage that they can be used to determine a binding site motif for any kinase, regardless of whether native substrates for that kinase have been identified. Furthermore, since the methods involve selection of peptides which are bound with high affinity by a kinase (i.e., low Kₘ), rather than peptides that represent the best substrates for that kinase (as defined by high Vₘₐₓ or high Vₘₐₓ/Kₘ ratios for phosphorylation of substrate), the amino acid sequence motif determined by the methods represents the optimal binding site for that kinase.

The binding affinity-based methods described herein require that a large quantity of kinase is contacted with the libraries of peptides. Preferably the kinase is immobilized on a solid surface, such as a resin bead, which permits thorough removal of unbound peptides, such as by washing, and recovery of kinase following removal of bound peptides (e.g., by alteration of salt concentration, pH, etc.).

In general one uses an oriented degenerate peptide library (ODPL, a partially degenerate library) to determine the kinase binding peptide motif. The orientation is provided by the inclusion of only one phosphorylatable amino acid (tyrosine, serine or threonine) in the peptide library. The phosphorylatable amino acid orients the peptides of the library in the kinase active site. Therefore, to maintain the orientation, the phosphorylatable amino acid is not included in the pool of amino acids for any of the degenerate amino acid positions in the peptides of the library. For example, in the degenerate peptide library described in the Examples, tyrosine occupies position 0, but is not permitted to occupy any of the other positions (e.g., −4, −3, −2, −1, +1, +2, +3, +4). Because of the orientation provided by the use of a single phosphorylatable peptide, the information gained from sequencing of the peptides selected for binding to the kinase will be meaningful, because each peptide will have bound to the kinase in the same orientation.

In some embodiments, the peptides that do not bind with high affinity to the kinase are washed away from immobilized kinase molecules. The higher affinity bound peptides subsequently can be eluted from kinase molecules using standard techniques (e.g., changing pH or salt concentration of the wash buffer). Alternatively, the peptides of the library can be labeled with a moiety that facilitates ready separation of kinase/peptide complexes from the reaction mixture, or separation of the eluted peptides after removal of unbound peptides. For example, the peptides can include a terminal biotin moiety. Avidin molecules can be coupled to a substrate (e.g., a bead, resin, dipstick, magnetic bead) and used to bind biotin-linked peptides or kinase/peptide complexes. The biotin-avidin binding pair is but one example of agents useful for removing the peptides. Other binding pairs known in the art include antibody-antigen pairs.

Following removal of unbound peptides and elution of bound peptides from the kinase, the previously bound peptides are sequenced according to standard methodologies. Preferably the peptides are sequenced by an automated peptide sequencer.

The peptides of the peptide library preferably are modified at the amino termini by the addition of amino acids that serve to confirm the peptides being sequenced (e.g., by automated Edman degradation) are peptides of the library. For example, as described here, the addition of a Met-Ala dipeptide at the N-terminus is suitable for this function. The amino-terminal added amino acids (i.e., non-degenerate amino acids) also are useful for quantitation of the peptides.

The peptides of the peptide library preferably are modified at the carboxy termini by the addition of amino acids that promote solubility of the peptide library. As shown herein, a poly-lysine tail can be used for this purpose. The carboxy termini of the library peptides also can include one or more amino acids that aid in the determination of peptide loss during sequencing. In the peptide libraries described herein, an alanine residue is joined to the carboxy terminal degenerate amino acid (i.e., the Ala is added at the +5 position) for this purpose, and the poly-lysine tail is added to the alanine residue. Other amino acid sequences that can function in these capacities can be substituted for those described herein.

In general, the amino- and carboxy-terminal amino acid modifications are not included in peptides that are used as inhibitors. Other amino acids can be added to the kinase inhibitory peptides identified by the methods of the invention, if desired, in accordance with standard methods of peptide synthesis. In addition, as described elsewhere herein, the inhibitory peptides can be joined to other compounds or moieties (such as peptides that enhance peptide translocation across membranes, for example, penetratin) to facilitate delivery of the inhibitory peptides.

An amino acid sequence motif for the binding site of a kinase can be determined from the most abundant amino acid residues at each degenerate position of the peptides. Upon sequencing of the peptides that bind to the kinase molecules, the abundance of an amino acid at a position in the peptide provides a preference value for each amino acid at each degenerate position. The preference value for a particular amino acid is determined by dividing the amount of the particular amino acid identified in a sequencing cycle by the average amount per amino acid in that cycle. This provides a raw value for the particular amino acid. To correct for bias in the library, it is preferred that the raw value is corrected by then dividing the raw value for each amino acid by the relative amount of that particular amino acid in the starting mixture. Amino acid residues that have a preference value of greater than 1.0 at a degenerate position are considered to be a part of the cleavage site motif. Higher preference values are preferred, e.g., 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, and so on, particularly preference values of greater than 1.5, and even more preferably preference values greater than 2.0. Thus one can select kinase binding site motifs based on the highest preference value at a particular peptide residue, or can select motifs based on a combination of two or more amino acids at a particular residue that have preference values above a certain cutoff score.

The peptides synthesized for the libraries can be of any size that is bound by kinases with high affinity for determination of binding site motifs. The size of the peptides can be determined empirically, although it is expected that a peptide length of 5-25 amino acids, e.g., 5, 6, 7, 8, 9, 10, 11, 12, 13, 14, 15, 16, 17, 18, 19, 20, 21, 22, 23, 24 and 25 amino acids, will work well for most applications of the methods described herein. Preferably, the peptides are 7-15 amino acids in length, most preferably 9-11 amino acids. Inhibitor peptides will generally be of a similar length, as typically the inhibitor is made by substituting a non-phosphorylatable amino acid for the phosphorylatable amino acid in the consensus binding peptide. It is possible, however, to use peptides that are longer if required or preferred for a particular application.

The substitution of a non-phosphorylatable amino acid for the phosphorylatable amino acid in the binding motif generally increases the inhibition of the kinase, because it cannot be phosphorylated and therefore is not released from the active site of the kinase. For tyrosine kinases, the phosphorylatable Tyr residue is substituted with a non-Tyr or Tyr derivative that is not phosphorylatable. Preferred substitutions include phenylalanine (Phe), which is most similar in structure among the natural amino acids, but is not phosphorylatable. For unnatural amino acids, preferred amino acids include halogenated Phe. See also Niu and Lawrence (1997). For serine and threonine kinases, alanine (Ala) is a preferred non-phosphorylatable substitution. For threonine kinases, valine (Val) is another preferred non-phosphorylatable substitution.

The peptides can incorporate natural and/or unnatural amino acids (also known as pseudo amino acids), and can be synthesized using standard solid-phase chemistries. For incorporation into libraries, it is preferred that the unnatural amino acids are able to form standard peptide bonds (i.e., have alpha amino groups), so that the library peptides can be sequenced by automated Edman degradation. If the unnatural amino acids are to be substituted for an amino acid in an identified peptide sequence, then the amino acid need not necessarily be cleavable for Edman degradation. For some binding motif determinations, it will be preferred that the library does not contain cysteine residues so that disulfide bonds are not formed. In general, it is preferred that the libraries used in the methods do not contain tryptophan (Trp) as this amino acid has a very low yield in automated Edman sequencers. If desired, one can substitute Trp into a consensus binding motif peptide by single amino acid replacements to determine if the consensus motif would be improved by the inclusion of Trp at certain positions.

Specific high affinity kinase inhibitors can be designed to incorporate the binding site motif determined as described herein. Many modifications to peptide structure are known that are useful in the preparation of kinase inhibitors. These include modified bonds, modified amino acids, and preferably substitution of the phosphorylatable amino acid with an amino acid that cannot be phosphorylated by the kinase the inhibitor is selected for (e.g., Tyr for Tyr-kinases) to prevent phosphorylation. Specific methods for synthesis and purification of the inhibitors are known in the art, and certain of these are described in more detail in the Examples below.

The potency of the candidate inhibitors in the inhibition of kinases can be determined initially in vitro, and their specificity for inhibition of a kinase can be evaluated by assaying for their ability to inhibit other kinases. Next, the ability of the candidate inhibitors to affect cellular processes mediated by the kinase can be determined.

As an approach for generating more potent inhibitors, new peptide libraries which incorporate “unnatural” amino acids can be screened. By expanding the repertoire of available residues, peptides are identified that bind kinases with high affinity, and corresponding inhibitors derived from such peptides are anticipated to have improved potency. Use of unnatural amino acids in the libraries requires slight modifications to standard protocols whereby libraries are analyzed by Edman degradation-based amino-terminal sequencing of the peptide mixtures. Quantitative analysis of peptides by Edman degradation is carried out by measuring the yield of the phenylthiohydantoin (PTH) adduct for a particular amino acid residue which is identified based on its retention time on an HPLC column. To incorporate unnatural amino acids into the mixture, their PTH adducts need simply have a retention time distinct from those of all other components in the library. A vast number of unnatural amino acids have been identified which are both commercially available in the Fmoc-protected form suitable for solid phase peptide synthesis and whose PTH adducts have unique retention times (see Liu et al., 2001).

A variety of unnatural amino acids are known to the skilled artisan, including unnatural analogs of phosphorylatable amino acids. For example, tyrosine-based unnatural amino acids include Fmoc-D,L-norTyr(tBu), Fmoc-L-norTyr(tBu), L-norTyr, Fmoc-L-m-Tyr(tBu), Fmoc-L-Tyr(4-CH2-OH), Boc-D, and L-Tyr(2,6-di-Me). Companies that produce unnatural amino acids include Amino Acid Analogues, Inc. (Hopkinton, Mass.), BACHEM Bioscience Inc. (Philadelphia, Pa.), BACHEM California Inc. (Torrance, Calif.) and The Peptide Laboratory (Bernicia, Calif.). Additional unnatural amino acids will be known to one of ordinary skill in the art.

Degenerate positions in unnatural amino acid-containing libraries can be of similar complexity as in natural amino acid containing libraries. Mixtures of unnatural and natural amino acids preferably include the optimal natural amino acid residue for each position to allow one to determine if any unnatural amino acid is an improvement over the natural one at a given position.

In some embodiments, it may be desired to prepare noncleavable peptides containing the binding motif sequences. For example, noncleavable peptide inhibitors may have a longer half life than standard peptides. Thus, for use as inhibitors, the peptides described herein preferably are non-hydrolyzable. To provide such peptides, one may select peptides from a library of non-hydrolyzable peptides, such as peptides containing one or more D-amino acids or peptides containing one or more non-hydrolyzable peptide bonds linking amino acids. Alternatively, one can determine kinase binding motifs and then synthesize non-hydrolyzable peptides or modify peptides as necessary to reduce the potential for hydrolysis by proteases. For example, the individual peptide bonds which are susceptible to proteolysis can be replaced with non-hydrolyzable peptide bonds by in vitro synthesis of the peptide.

Many non-hydrolyzable peptide bonds are known in the art, along with procedures for synthesis of peptides containing such bonds. Non-hydrolyzable bonds include -psi[CH₂NH]— reduced amide peptide bonds, -psi[COCH₂]— ketomethylene peptide bonds, -psi[CH(CN)NH]— (cyanomethylene)amino peptide bonds, -psi[CH₂CH(OH)]— hydroxyethylene peptide bonds, -psi[CH₂O]— peptide bonds, and -psi[CH₂S]— thiomethylene peptide bonds.

Nonpeptide analogs of peptides, e.g., those which provide a stabilized structure or lessened biodegradation, are also contemplated. Peptide mimetic analogs can be prepared based on a binding motif sequence by replacement of one or more amino acid residues by nonpeptide moieties. Preferably, the nonpeptide moieties permit the peptide mimetic to retain its natural confirmation, or stabilize a preferred, e.g., bioactive, confirmation. Particularly preferred are those mimetics that resist protease cleavage. One example of methods for preparation of nonpeptide mimetic analogs from peptides is described in Nachman et al., Regul. Pept. 57:359-370 (1995). “Peptide,” as used herein, embraces all of the foregoing.

The peptides (binding peptides and inhibitors derived from the binding peptides) described herein can be linked to a molecule that facilitates or mediates transport across plasma membranes. Numerous examples of such molecules are known in the art, including peptides derived from the third alpha helix of the homeodomain of Drosophila Antennapedia (penetratins; Derossi et al., 1998; Fischer et al., 2000); HIV-1 Tat protein and derivatives thereof (e.g., D-amino acid and arginine-substituted Tat; Futaki et al., 2001); herpes simplex virus VP22; transportan, a chimeric peptide constructed from the 12 N-terminal residues of galanin in the N-terminus with the 14-residue sequence of mastoparan in the C-terminus and a connecting lysine (Lindgren et al., 2000); arginine-rich peptides, including RNA binding peptides derived from virus proteins such as HIV-1 Rev and the flock house virus coat proteins, and DNA binding segments of leucine zipper proteins such as c-Fos, c-Jun and yeast transcription factor GCN4 (Futaki et al., 2001); synthetic peptides, including polylysine and polyarginine; and amphipathic peptide carriers such as Pep-1 (Morris et al., 2001).

The binding peptides and kinase inhibitors described herein are useful for screening compounds and libraries of compounds for kinase inhibitory activity. High throughput screening of known compounds and libraries of compounds can be performed using these peptides according to known methodologies.

The binding peptides and kinase inhibitors described herein also are useful for validating kinases as targets for therapeutic intervention. For example, a particular condition may be the result of excessive activity of a particular kinase. Application of binding peptides and inhibitors to specifically inhibit that particular kinase can confirm the importance of the kinase in the etiology of the condition. Thus the peptides identified through use of the methods of the invention can be used in cellular and animal model systems of disease to validate therapeutic targets, by specifically inhibiting a kinase and measuring or observing the effects on the model system. Specific inhibition of the kinase can be confirmed by biochemical assays, such as the protein substrate phosphorylation assays described in the Examples.

A wide variety of assays for pharmacological agents are provided, including labeled in vitro kinase assays, cell-based kinase assays, etc. For example, in vitro kinase assays are used to rapidly examine the effect of candidate pharmacological agents on the phosphorylation of a substrate by a specific kinase. The candidate pharmacological agents can be derived from, for example, combinatorial peptide or small molecule libraries. Convenient reagents for such assays are known in the art.

Peptides used in the methods of the invention are added to an assay mixture as an isolated peptide. Peptides can be produced recombinantly, or isolated from biological extracts, but preferably are synthesized in vitro. Peptides encompass chimeric proteins comprising a fusion of a peptide having a particular binding site motif with one or more other polypeptides, e.g., mediators of plasma membrane transport. Peptides may also be labeled with detectable compound(s) to provide a means of readily detecting whether the peptide binds to a kinase, e.g., by immunological recognition or by fluorescent labeling.

A typical assay mixture includes a peptide having a kinase binding site motif and a candidate pharmacological agent. Typically, a plurality of assay mixtures are run in parallel with different agent concentrations to obtain a different response to the various concentrations. Typically, one of these concentrations serves as a negative control, i.e., at zero concentration of agent or at a concentration of agent below the limits of assay detection. Candidate agents encompass numerous chemical classes, although typically they are organic compounds. Preferably, the candidate pharmacological agents are small organic compounds, i.e., those having a molecular weight of more than 50 yet less than about 2500. Candidate agents comprise functional chemical groups necessary for structural interactions with polypeptides (e.g., kinase binding sites), and typically include at least an amine, carbonyl, hydroxyl or carboxyl group, preferably at least two of the functional chemical groups and more preferably at least three of the functional chemical groups. The candidate agents can comprise cyclic carbon or heterocyclic structure and/or aromatic or polyaromatic structures substituted with one or more of the above-identified functional groups. Candidate agents also can be biomolecules such as peptides (preferably non-hydrolyzable, and/or lacking an amino acid phosphorylatable by the kinase it inhibits), saccharides, fatty acids, sterols, isoprenoids, purines, pyrimidines, derivatives or structural analogs of the above, or combinations thereof and the like. Where the agent is a nucleic acid (i.e., aptamer), the agent typically is a DNA or RNA molecule, although modified nucleic acids having non-natural bonds or subunits are also contemplated.

Candidate agents are obtained from a wide variety of sources including libraries of synthetic or natural compounds. For example, numerous means are available for random and directed synthesis of a wide variety of organic compounds and biomolecules, including expression of randomized oligonucleotides, random or non-random peptide libraries, synthetic organic combinatorial libraries, phage display libraries of random peptides, and the like. Alternatively, libraries of natural compounds in the form of bacterial, fungal, plant and animal extracts are available or readily produced. Additionally, natural and synthetically produced libraries and compounds can be readily be modified through conventional chemical, physical, and biochemical means. Further, known pharmacological agents may be subjected to directed or random chemical modifications such as acylation, alkylation, esterification, amidification, etc. to produce structural analogs of the agents. Based on the identification of peptides of specific sequence using the methods of the invention, agents can include modifications of the specific sequence, such as the site specific incorporation of unnatural amino acids.

A variety of other reagents also can be included in the mixture. These include reagents such as salts, buffers, neutral proteins (e.g., albumin), detergents, etc. which may be used to facilitate optimal protein-protein and/or protein-nucleic acid binding. Such a reagent may also reduce non-specific or background interactions of the reaction components. Other reagents that improve the efficiency of the assay such as nuclease inhibitors, antimicrobial agents, and the like may also be used. Kinase substrates also can be included in the assays to monitor kinase activity.

The mixture of the foregoing assay materials is incubated under conditions whereby, but for the presence of the candidate pharmacological agent, a kinase phosphorylates a substrate, or specifically binds a peptide (for identifying compounds that compete with known inhibitors). The order of addition of components, incubation temperature, time of incubation, and other parameters of the assay may be readily determined. Such experimentation merely involves optimization of the assay parameters, not the fundamental composition of the assay. Incubation temperatures typically are between 4° C. and 40° C. Incubation times preferably are minimized to facilitate rapid, high throughput screening, and typically are between 1 minute and 10 hours.

After incubation, the presence or absence of phosphorylation or binding of a substrate is detected by any convenient method available to the user. For cell free binding type assays, a separation step may be used to separate bound from unbound components. The separation step may be accomplished in a variety of ways. Conveniently, at least one of the components is immobilized on a solid substrate, from which the unbound components may be easily separated. The solid substrate can be made of a wide variety of materials and in a wide variety of shapes, e.g., microtiter plate, microbead, dipstick, resin particle, etc. The substrate preferably is chosen to maximum signal to noise ratios, primarily to minimize background binding, as well as for ease of separation and cost.

Separation may be effected for example, by removing a bead or dipstick from a reservoir, emptying or diluting a reservoir such as a microtiter plate well, rinsing a bead, particle, chromatographic column or filter with a wash solution or solvent. The separation step preferably includes multiple rinses or washes. For example, when the solid substrate is a microtiter plate, the wells may be washed several times with a washing solution, which typically includes those components of the incubation mixture that do not participate in specific binding or interaction such as salts, buffer, detergent, non-specific protein, etc. Where the solid substrate is a magnetic bead, the beads may be washed one or more times with a washing solution and isolated using a magnet.

Detection may be effected using any convenient method. The kinase phosphorylation or binding typically alters a directly or indirectly detectable product. In the assays, one of the components usually comprises, or is coupled to, a detectable label. A wide variety of labels can be used, such as those that provide direct detection (e.g., radioactivity, luminescence, optical or electron density, etc). or indirect detection (e.g., epitope tag such as the FLAG epitope, enzyme tag such as horseradish peroxidase, etc.). The label may be bound to a inhibitor as described elsewhere herein or to the candidate pharmacological agent.

A variety of methods may be used to detect the label, depending on the nature of the label and other assay components. For example, the label may be detected while bound to the solid substrate or subsequent to separation from the solid substrate. Labels may be directly detected through optical or electron density, radioactive emissions, nonradiative energy transfers, etc. or indirectly detected with antibody conjugates, streptavidin-biotin conjugates, etc. Methods for detecting the labels are well known in the art.

Thus the present invention includes assays for identifying compositions having the ability to inhibit a kinase directly by binding to the kinase. The methods can be automated, in which case they are carried out in an apparatus which is capable of delivering a reagent solution to a plurality of predetermined compartments of a vessel and measuring the change in a detectable molecule in the predetermined compartments.

Inhibitors of kinases identified by the methods described herein are useful to treat diseases or conditions that result from excessive or unwanted kinase activity, including cancer, inflammatory diseases, autoimmune diseases, transplant rejection, etc. For treatment of such conditions, an effective inhibitory amount of a kinase inhibitor is administered to a subject. As used herein, a subject is a mammal, preferably a human, non-human primate, cow, horse, pig, sheep, goat, dog, cat, or rodent. In all embodiments human subjects are preferred.

The inhibitors also can be used in diagnostic applications, to detect specific kinases. For example, the involvement of a specific kinase in disease can be identifies detected in a subject, in cellular or animal models of the disease, or in a biological sample of the subject.

Inhibitors and other compounds that incorporate kinase binding site sequence motifs can be administered as part of a pharmaceutical composition. Such a pharmaceutical composition may include the compounds in combination with any standard physiologically and/or pharmaceutically acceptable carriers which are known in the art. The compositions should be sterile and contain a therapeutically effective amount of the inhibitor peptide or other therapeutic compound in a unit of weight or volume suitable for administration to a patient. The term “pharmaceutically acceptable” means a non-toxic material that does not interfere with the effectiveness of the biological activity of the active ingredients. The term “physiologically acceptable” refers to a non-toxic material that is compatible with a biological system such as a cell, cell culture, tissue, or organism. The characteristics of the carrier will depend on the route of administration. Physiologically and pharmaceutically acceptable carriers include diluents, fillers, salts, buffers, stabilizers, solubilizers, and other materials which are well known in the art.

When used therapeutically, the compounds of the invention are administered in therapeutically effective amounts. In general, a therapeutically effective amount means that amount necessary to delay the onset of, inhibit the progression of, or halt altogether the particular condition being treated. Therapeutically effective amounts specifically will be those which desirably influence kinase activity. Generally, a therapeutically effective amount will vary with the subject's age, and condition, as well as the nature and extent of the disease in the subject, all of which can be determined by one of ordinary skill in the art. The dosage may be adjusted by the individual physician, particularly in the event of any complication. A therapeutically effective amount typically varies from 0.01 ng/kg to about 1000 μg/kg, preferably from about 0.1 ng/kg to about 200 μg/kg and most preferably from about 0.2 ng/kg to about 20 μg/kg, in one or more dose administrations daily, for one or more days.

The therapeutics of the invention can be administered by any conventional route, including injection or by gradual infusion over time. The administration may, for example, be oral, intravenous, topical, intracranial, intraperitoneal, intramuscular, intracavity, intrarespiratory, subcutaneous, or transdermal. The route of administration will depend on the composition of a particular therapeutic preparation of the invention and its intended use.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Other delivery systems can include time-release, delayed release or sustained release delivery systems. Such systems can avoid repeated administrations of the active compounds of the invention, increasing convenience to the subject and the physician. Many types of release delivery systems are available and known to those of ordinary skill in the art. They include polymer based systems such as polylactic and polyglycolic acid, polyanhydrides and polycaprolactone; nonpolymer systems that are lipids including sterols such as cholesterol, cholesterol esters and fatty acids or neutral fats such as mono-, di and triglycerides; hydrogel release systems; silastic systems; peptide based systems; wax coatings, compressed tablets using conventional binders and excipients, partially fused implants and the like. In addition, a pump-based hardware delivery system can be used, some of which are adapted for implantation.

A long-term sustained release implant also may be used. “Long-term” release, as used herein, means that the implant is constructed and arranged to deliver therapeutic levels of the active ingredient for at least 30 days, and preferably 60 days. Long-term sustained release implants are well known to those of ordinary skill in the art and include some of the release systems described above. Such implants can be particularly useful in treating conditions characterized by unwanted kinase activity by placing the implant near portions of a subject affected by such activity, thereby effecting localized, high doses of the compounds of the invention.

### EXAMPLES

Experimental Procedures

Peptide Library

A tyrosine-oriented degenerate peptide library of general sequence Met-Ala-X-X-X-X-Tyr-X-X-X-X-Ala-Lys-Lys-Lys (SEQ ID NO: 1), theoretical degeneracy=6.9×10⁹, was synthesized using N-α-FMOC-protected amino acids and standard BOP/HOBt coupling chemistry. X represents all amino acids except Trp, Cys, or Tyr. The Met-Ala sequence at the amino terminus of the Tyr-fixed peptide library was included to verify that peptides from this mixture are being sequenced and to quantify the peptides present. Ala at position +5 provides an estimate of peptide loss during sequencing. The poly(Lys) tail prevents wash-out during sequencing and improves the solubility of the library mixture.

Library Screening

Human ZAP-70-glutathione S-transferase (GST)-fused protein was expressed in Sf9 cells using baculovirus. In this study, we used GST-ZAP-70 expressed in Sf9 cells without coexpression of Lck for library screening and kinase assays because the substrate selectivity of partially activated ZAP-70 is higher than that of fully activated ZAP-70 in peptide library screening for their specific substrate motifs (unpublished results). ZAP-70-GST fused protein (600 μg) was purified by using glutathione-agarose beads. The fused protein bound to the beads was packed in a small column and incubated with 450 μg of the peptide library in a solution containing 100 μM ATP, 1 mM DTT, 5 mM EDTA, and 50 mM Tris (pH 7.4), for 10 min at room temperature. Unbound peptides were removed by rapid washing, and bound peptides were eluted with 30% acetic acid for 10 min at room temperature, dried overnight on a Speed-Vac apparatus, resuspended in H₂O, and sequenced on an Applied Biosystems model 477A protein sequencer (Applied Biosystems, Foster City, Calif.) (Songyang et al., 1994).

The abundance of each amino acid at a given cycle in the recovered peptides from the GST control column was subtracted from the data obtained from ZAP-70-GST affinity column to correct the background. To calculate the relative preference for amino acids at each degenerate position, the corrected quantities of amino acids were then compared with those in the starting mixture to calculate the ratios of abundance of amino acids.

Kinase Reaction

Synthetic peptides, tubulin, or GST-band III (construct kindly provided by A. Weiss, University of California, San Francisco) (Zhao and Weiss, 1996) were phosphorylated by ZAP-70-GST-fused protein in the presence or absence of the indicated amount of inhibitory peptides in the kinase buffer (10 mM MnCl₂, 50 mM Tris [pH 7.4], 1 mM DTT), 100 μM ATP, and 5 μCi [γ-³²P]ATP for 5 min (or 10 min for GST-band-III) at 30° C. The amount of radioactivity incorporated into substrate peptide was determined using the phosphocellulose assay. Lck-GST-fused protein was purified from Sf9 cells. Myelin basic protein was phosphorylated by Lck-GST-fused protein in the presence or absence of inhibitory peptides in the kinase buffer (5 mM MnCl₂, 5 mM MgCl₂, 25 mM Tris [pH 7.4]), 50 μM ATP, and 5 μCi [γ-³²P]ATP for 5 min at 30° C. Myc-tagged Syk kinase was expressed and purified from COS-7 cells (pEBM-Syk-myc was prepared from original myc-tagged Syk cDNA provided by R. Geahlen, Purdue University). GST-band III was phosphorylated by myc-tagged Syk in the presence or absence of inhibitory peptides in the kinase buffer (10 mM MnCl₂, 0.1% Triton X-100, 50 mM Tris [pH 7.2]), 250 μM ATP, and 5 μCi [γ-³²P]ATP for 10 min at 30° C. Phosphorylated tubulin, GST-band III, and myelin basic protein were separated on SDS-PAGE, visualized by autoradiography, and quantitated using a molecular imager (Bio-Rad, Hercules, Calif.).

For each experimental condition, values for control reactions lacking substrate peptide were subtracted as blanks. In all assays to determine kinetic parameters, reaction rates were linear with respect to time for all conditions of peptide, and less than 10% of the peptide substrate was phosphorylated. Kₘ and Vₘₐₓ were determined by nonlinear regression analysis using KaleidaGraph (Abelbeck Software, Reading, Pa.). Each experiment consisted of 6-7 substrate concentrations.

Tyr Phosphorylation of PLCγ1 or LAT in Jurkat Cells

Ten million Jurkat cells, suspended in 500 μl of serum-free RPMI1640, were incubated with penetratin, penetratin peptide, or DMSO at 37° C. for 30 min. Peptide pretreated cells were treated with 1 μg of the anti-CD3ε mAb (OKT3) at 4° C. for 10 min, followed by another 10 min incubation, in the presence of 5 μg of rabbit anti-mouse Ig. Cells were activated by shifting the temperature to 37° C. for 2 min and subsequently lysed in 1% NP40 lysis buffer. Lysates were immunoprecipitated with either the 4G10 anti-phosphotyrosine mAb (Upstate Biotechnology, Inc., Lake Placid, N.Y.), or with anti-PLCγ1 antibody (Upstate Biotechnology, Inc.), and immunoblotted with the HRP-conjugated anti-phosphotyrosine Ab, RC20H (Transduction Laboratories, Lexington, Ky.). The membrane was finally visualized by chemiluminescence (NEN Life Science Products, Boston, Mass.). Each blot was stripped and reprobed with anti-PLCγ1 antibody, or with anti-LAT antibody.

IL-2 Reporter Gene Analysis

Jurkat cells were electroporated at 250V, 800 μF settings (Invitrogen Life Technologies, Carlsbad, Calif.), in the presence of 10 μg of plasmid carrier DNA (pCAGGS vector), 100 ng of Renilla luciferase control vector (pRL null), and 2.5 μg of a luciferase reporter gene whose transcriptional activity is controlled by the human IL-2 promoter (IL-2-Luc). Transfectants were treated with 10 μM of each peptides or DMSO. Treated cells were stimulated with 10 ng/ml of PMA plus 2 μM of ionomycin, or plate-bound anti-CD3ε mAb (OKT3) and 10 ng/ml of PMA at 37° C. for 2 hr. Luciferase activities from lysates were measured using a Dual-Luciferase Reporter System (Promega, Madison, Wis.) and Monolight 2010 luminometer (Analytical Luminescence Laboratory, San Diego, Calif.). Relative values were normalized against the activities of the coexpressed Renilla luciferase.

Results and Discussion

In order to identify high-affinity inhibitors of the ZAP-70 protein Tyr kinase, we screened a Tyr-oriented peptide library by affinity purification rather than by catalytic conversion. The peptide library used contained the sequence Met-Ala-X-X-X-X-Tyr-X-X-X-X-Ala-Lys-Lys-Lys (SEQ ID NO: 1) where X indicates all amino acids except Trp, Cys, or Tyr. The predicted degeneracy of this library is 17⁸, which is about 6.9 billion. Screening was performed in the presence of 100 μM ATP but in the absence of Mg²⁺ to prevent peptide phosphorylation and turnover. The subset of peptides that preferentially bound to ZAP-70 was eluted and sequenced as a batch using an automated Edman sequencer. The relative abundance of individual amino acids at positions N-terminal or C-terminal to the orienting Tyr residue reflects the relative abundance of high-affinity peptides that contain these same amino acids (Songyang et al., 1994).

TABLE 1
Optimal Binding Motif for ZAP-70

| Posi- | | | | | | | | |
|---|---|---|---|---|---|---|---|---|
| tion | | | | | | | | |
| −4 | −3 | −2 | −1 | 0 | 1 | 2 | 3 | 4 |
| K | L(1.8) | I(2.0) | L(2.0) | Y | L | L(2.1) | L(2.7) | L |
| (2.0) | | | | | (2.6) | | | (2.6) |
| Q | I(1.7) | L(1.8) | I(1.8) | | I | T(2.1) | T(1.8) | T |
| (1.6) | | | | | (1.6) | | | (1.8) |
| I | K(1.6) | | T(1.8) | | T | I(1.9) | I(1.7) | I |
| (1.5) | | | | | (1.5) | | | (1.6) |
| | Q(1.6) | | | | V(1.5) | | Q(1.5) | Q(1.5) |

Human ZAP-70 was expressed in Sf9 cells using baculovirus. A kinase binding library with the sequence Met-Ala-X-X-X-X-Y-X-X-X-X-Ala-Lys-Lys-Lys (SEQ ID NO: 1; where X indicates any amino acids apart from Trp, Cys, or Tyr) was used to screen the ZAP-70 binding motif. Approximately 600 μg of ZAP-70-GST-fused protein bound to glutathione beads was incubated with 450 μg of the peptide library in a solution containing 100 μM ATP, 1 mM DTT, 5 mM EDTA, and 50 mM Tris (pH 7.4) for 10 min at room temperature. Following extensive washing, bound peptides were eluted and sequenced. Values in parentheses indicate the relative selectivities for the amino acids as described previously (Songyang et al., 1994); amino acids with values less than 1.5 are omitted. Letters in italics indicate amino acids that are strongly selected. The one-letter amino acid code is used as follows: A, Ala; E, Glu; F, Phe; I, Ile; K, Lys; L, Leu; M, Met; Q, Gln; R, Arg; T, Thr; V, Val; W, Trp; and Y, Tyr. ZAP-70 was evaluated twice; representative values are shown. The binding motif peptide sequence is SEQ ID NO: 2; the predicted optimal binding peptide (Lys-Leu-Ile-Leu-Tyr-Leu-Leu-Leu-Leu) is SEQ ID NO: 4.

The amino acids selected at each position relative to the orienting Tyr are presented in Table 1. Leu or Ile was strongly selected at all positions except −4, where Lys was preferred. The strong selection for Leu at the +3 position (selectivity value 2.7) is consistent with consensus optimal substrates determined for both cytosolic and receptor-type protein Tyr kinases where Leu, Ile, or Phe is typically selected at +3 (Songyang et al., 1995). The selection for Leu, Ile, or Thr at −1 is also consistent with optimal substrates for cytosolic protein Tyr kinases (Songyang et al., 1995). However, the selection for hydrophobic residues at −2 and −3 and for Lys at −4 is at odds with optimal substrates for protein Tyr kinases where Glu is typically preferred at all these positions. In fact, the predicted optimal substrate for ZAP-70 (Glu-Glu-Glu-Glu-Tyr-Phe-Phe-Ile-Ile (SEQ ID NO: 3); unpublished results) differs strikingly from the predicted optimal binding peptide (Lys-Leu-Ile-Leu-Tyr-Leu-Leu-Leu-Leu (SEQ ID NO: 4); Table 1).

Kinetic analyses revealed that the predicted optimal ZAP-70 binding peptide acts as a low Kₘ, low Vₘₐₓ substrate. We synthesized the optimal ZAP-70 substrate (peptide 1 (SEQ ID NO: 5); Table 2) and the predicted optimal ZAP-70 binding peptide (peptide 2 (SEQ ID NO: 6); Table 2), both in the context of the C-terminal Ala-Lys-Lys-Lys motif (to ensure solubility; SEQ ID NO: 7). When compared to the optimal substrate peptide, peptide 2 had a much lower Vₘₐₓ (14-fold lower), but it also had a lower Kₘ (26 μM versus 40 μM), suggesting that it binds with higher affinity but turns over more slowly. To determine whether the unusual selection for Lys at −4 is relevant, we made a third peptide identical to peptide 2 except for a Glu substituted for Lys at −4. This peptide (peptide 3; SEQ ID NO: 8) had a 7-fold higher Km than peptide 2, indicating that the Lys at −4 is important for high-affinity binding (Table 2).

TABLE 2
Kinetic Parameters for the Phosphorylation of Synthetic Peptides by ZAP-70

| Peptide | | SEQ ID | | | |
|---|---|---|---|---|---|
| No. | Sequence | NO | Vₘₐₓ(nmo1/min/mg) | Km (μM) | Vₘₐₓ/Kₘ |
| 1 | EEEEYFFII AKKIK | 5 | 80 | 40 | 2 |
| 2 | KLILYLLLL AKKK | 6 | 6 | 26 | 0.22 |
| 3 | ELILYLLLL AKKK | 8 | 18 | 188 | 0.09 |

Three synthetic peptides, EEEEYFFII AKKK (peptide 1; SEQ ID NO: 5), KLILYLLLL AKKK (peptide 2; SEQ ID NO: 6), and ELILYLLLL AKKK (peptide 3; SEQ ID NO: 8), were phosphorylated by ZAP-70-GST-fused protein in the kinase buffer (10 mM MnCl₂, 50 mM Tris (pH 7.4), 1 mM DTT), 100 μM ATP, and 5 μCi [γ-³²P]ATP for 5 min at 30° C. The amount of radioactivity incorporated was determined using the phosphocellulose assay. Values are the average of three independent experiments.

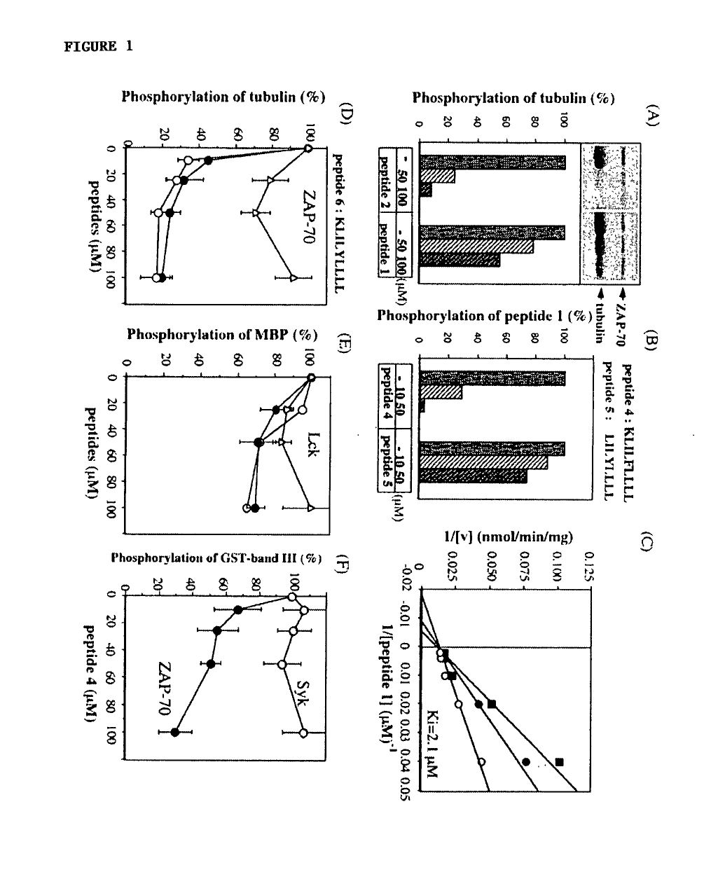

Consistent with its high affinity as judged by Kₘ, measurements, peptide 2 acted as a potent competitive inhibitor of ZAP-70. For inhibition studies, tubulin, a well-characterized in vitro substrate of ZAP-70 (Isakov et al., 1996), was used. Peptide 2 was much more effective than peptide 1 in inhibiting tubulin phosphorylation by ZAP-70 (FIG. 1A). To further test whether the inhibition occurred at the catalytic site (versus inhibition by binding of phosphopeptide product at SH2 domains), we utilized peptide 1 as the substrate and investigated inhibition with a shorter version of the optimal binding motif in which the Tyr was substituted by Phe (peptide 4 (SEQ ID NO: 9), FIG. 1B). This peptide, which cannot be phosphorylated, was an even more effective inhibitor than peptide 2 with 70% inhibition occurring at 10 μM. This result argues for inhibition at the catalytic pocket rather than via SH2 domains. Further support for this conclusion is provided by Lineweaver-Burke plots utilizing peptide 1 as substrate and peptide 4 as inhibitor (FIG. 1C). This analysis indicated that peptide 4 is a competitive inhibitor with a K_{I} of 2 μM. Finally, deletion of the Lys at position −4 (peptide 5 (SEQ ID NO: 10), FIG. 1B) dramatically reduced the inhibitory ability. These results indicate that the optimal ZAP-70 binding peptide predicted by the peptide library screen acts as a high-affinity inhibitor at the catalytic site.

As shown in FIG. 1, peptide 4 inhibited ZAP-70 more than 50% at concentrations below 20 μM (FIG. 1D). However, it had only a modest effect on Lck Tyr kinase activity even at 100 μM (FIG. 1E). Peptide 5, lacking the Lys at −4, was less effective as an inhibitor of both kinases. A comparison of the ZAP-70 inhibitory effects of peptide 4 and peptide 6 (SEQ ID NO: 4; identical to peptide 4 except for Tyr replacing the Phe) indicated that substituting Tyr for Phe has little effect on peptide binding to ZAP-70 (FIG. 1D). This result is in contrast to results that we and others have observed for peptide inhibitors of Src where substitution of Tyr for Phe in the context of optimal substrates (rather than optimal binding peptides) considerably reduces the inhibitory effect. Some substrate-based inhibitors of Tyr kinases have been recently published (Niu and Lawrence, 1997; Alfaro-Lopez et al., 1998; McMurray et al., 1998). The marked differences between the optimal substrate and optimal binding motif of ZAP-70, at position −1 to −4, especially at position −4, may compensate for the loss of the phenolic hydroxy group of the Tyr residue. To further confirm the specificity of peptide 4 for ZAP-70, we evaluated its effect on Syk, a Tyr kinase closely related to, and classified in the same family as, ZAP-70. As shown in FIG. 1F, peptide 4 significantly inhibited the activity of ZAP-70 but not Syk in a dose-dependent manner using GST-band III as a common substrate.

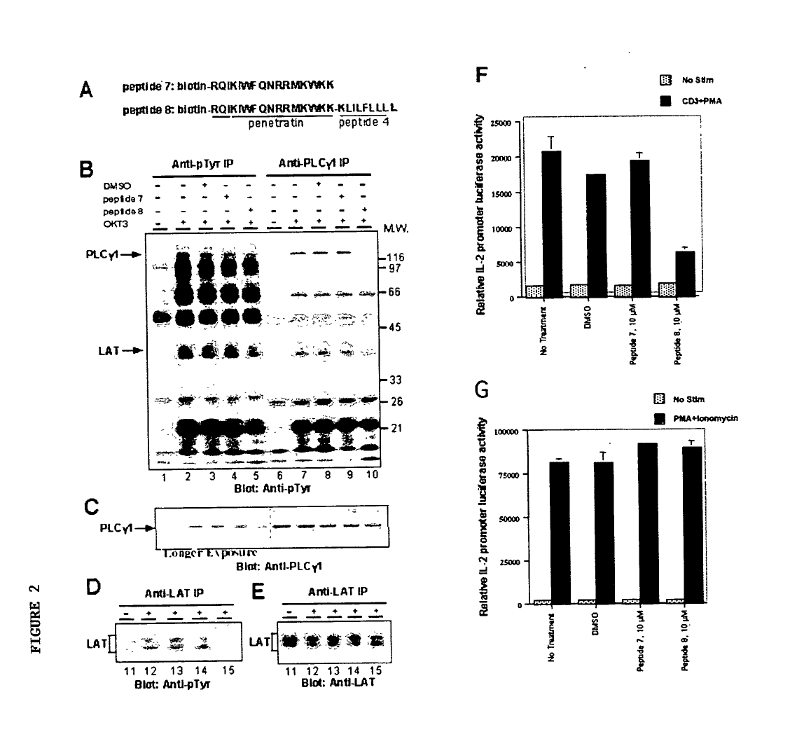

We next investigated the ability of the ZAP-70 inhibitor to block T cell responses. To deliver peptide 4 to the interior of T cells, we took advantage of the penetratin peptide (RQIKIWFQNRRMKWKK (SEQ ID NO: 11); peptide 7), which has been shown to mediate transport of associated peptides across the plasma membrane (Williams et al., 1997; Derossi et al., 1998). We made a chimeric peptide with penetratin at the N terminus and peptide 4 at the C terminus (peptide 8 (SEQ ID NO: 12); FIG. 2A). As a control, we used penetratin lacking peptide 4 (peptide 7; FIG. 2A). We first examined the effects of peptides 7 and 8 on anti-CD3-dependent tyrosine phosphorylation in T cells. CD3 cross-linking is known to stimulate Tyr-phosphorylation of a large set of proteins, and peptide 8 had little effect on most of these proteins (FIG. 2B; lane 5 versus control lanes 2, 3, and 4). However, peptide 8 specifically diminished the phosphorylation levels of proteins whose migration positions are consistent with the known ZAP-70 kinase substrates—PLCγ1 and LAT (FIG. 2B; lane 5). Anti-phosphoTyr blots of anti-PLCγ1 and anti-LAT immunoprecipitates confirmed that peptide 8 reduced the Tyr phosphorylation of PLCγ1 (FIG. 2B; lane 10) and LAT (FIG. 2D; lane 15). An anti-PLCγ1 blot of anti-phosphoTyr immunoprecipitates also confirmed the above conclusion (FIG. 2C; longer exposure). Reprobing of the membranes revealed that peptide 8 did not affect the amount of PLCγ1 and LAT precipitated (FIG. 2C, FIG. 2E). These observations indicate that peptide 8 specifically inhibited ZAP-70 activity in intact cells while having little effect on upstream Tyr phosphorylation attributed to src-family Tyr kinases, Lck or Fyn.

Finally, we investigated the ability of peptide 8 to block transcriptional activation using a reporter construct for the IL-2 gene. This reporter can be induced by phorbol ester plus ionomycin via a pathway that circumvents ZAP-70 (FIG. 2G) or by anti-CD3 plus phorbol ester via a ZAP-70-dependent pathway (FIG. 2F) (Qian and Weiss, 1997; van Leeuwen and Samelson, 1999). As shown in FIG. 2F, 10 μM of peptide 8 caused more than 70% inhibition of gene expression when using anti-CD3 plus phorbol ester as stimulant. However, peptide 8 had no effect on gene expression in response to phorbol ester plus ionomycin, indicating that the effect was specific to the CD3-ZAP-70 pathway. The penetratin peptide alone (peptide 7) had no significant effect on either pathway. Taken together, these observations demonstrate that the cell-permeable peptide 8 can specifically inhibit the signal transduction pathway mediated by ZAP-70 in intact T cells.

In summary, we have used an affinity-based peptide library screening procedure to determine a high-affinity and high-specificity ZAP-70 inhibitor. We show that a membrane-permeant version of this peptide can specifically inhibit ZAP-70 in intact T cells and thereby block CD3-dependent gene regulation. Previous attempts to design peptide or peptide-mimetic inhibitors of kinase catalytic sites have relied on optimal peptide substrates. Here we show that the optimal binding peptide for ZAP-70 differs considerably from the optimal substrate and would not have been found in a screen for substrates because of its low turnover rate. The high-affinity binding probably contributes to the low turnover because of a slow rate of release. These studies extend the range of experimental approaches for probing ZAP-70 function in vivo. Also, the method presented here is widely applicable for the design of highly selective inhibitors for other protein kinases and for the elucidation of their specific roles in vivo.

### References

Alfaro-Lopez, J., Yuan, W., Phan, B. C., Kamath, J., Lou, Q., Lam, K. S., and Hruby, V. J. J. (1998). Discovery of a novel series of potent and selective substrate-based inhibitors of p60c-src protein tyrosine kinase: conformational and topographical constraints in peptide design. Med. Chem. 41, 2252-2260.

Derossi, D., Chassaing, G., and Prochiantz, A. (1998). Trojan peptides: the penetratin system for intracellular delivery Trends. Cell Biol. 8, 84-87.

Fischer, P. M., Zhelev, N. Z., Wang, S., Melville, J. E., Fahraeus, R., Lane, D. P. (2000) Structure-activity relationship of truncated and substituted analogues of the intracellular delivery vector Penetratin. J. Pept. Res. 55(2), 163-172.

Futaki, S., Suzuki, T., Ohashi, W., Yagami, T., Tanaka, S., Ueda, K., Sugiura, Y. (2001) Arginine-rich peptides. An abundant source of membrane-permeable peptides having potential as carriers for intracellular protein delivery. J. Biol. Chem. 276(8), 5836-5840.

Isakov, N., Wange, R. L., Watts, J. D., Aebersold, R., and Samelson, L. E. (1996). Purification and characterization of human ZAP-70 protein-tyrosine kinase from a baculovirus expression system. J. Biol. Chem. 271, 15753-15761.

Lindgren, M., Gallet, X., Soomets, U., Hallbrink, M., Brakenhielm, E., Pooga, M., Brasseur, R., Langel, U. (2000) Translocation properties of novel cell penetrating transportan and penetratin analogues. Bioconjug. Chem. 11(5), 619-26.

Liu, R. and Lam, K. S. (2001) Automatic Edman microsequencing of peptides containing multiple unnatural amino acids. Anal. Biochem. 295, 9-16.

McMurray, J. S., Budde, R. J., Ke, S., Obeyesekere, N. U., Wang, W., Ramdas, L., and Lewis, C. A. (1998). Cyclic peptides as probes of the substrate binding site of the cytosolic tyrosine kinase, pp60c-src. Arch. Biochem. Biophys. 355, 124-130.

Morris, M. C., Depollier, J., Mery, J., Heitz, F., Divita, G. (2001) A peptide carrier for the delivery of biologically active proteins into mammalian cells. Nat. Biotechnol. 19(12), 1173-1176.

Nishikawa, K., Toker, A., Johannes, F. J., Songyang, Z., and Cantley, L. C. (1997). Determnination of the specific substrate sequence motifs of protein kinase C isozymes. J. Biol. Chem. 272, 952-960.

Niu, J., and Lawrence, D. S. (1997). Nonphosphorylatable tyrosine surrogates. Implications for protein kinase inhibitor design. J. Biol. Chem. 272, 1493-1499.

Qian, D. A., and Weiss, A. (1997). T cell antigen receptor signal transduction. Curr. Opin. Cell Biol. 9, 205-212.

Songyang, Z., Blechner, S., Hoagland, N., Hoekstra, M. F., Piwnica-Worms, H., and Cantley, L. C. (1994). Use of an oriented peptide library to determine the optimal substrates of protein kinases. Curr. Biol. 4, 973-982.

Songyang, Z., Carraway, K. L., III, Eck, M. J., Harrison, S. C., Feldman, R. A., Mohammadi, M., Schlessinger, J., Hubbard, S. R., Smith, D. P., Eng, C., et al. (1995). Catalytic specificity of protein-tyrosine kinases is critical for selective signalling. Nature 373, 536-539.

Songyang, Z., Lu, K. P., Kwan, Y., Tsai, L., Filhol, O., Cochet, C., Soderling, T. R., Bartleson, C., Graves, D. J., DeMaggio, A. J., et al. (1996). A structural basis for substrate specificities of protein Ser/Thr-kinases: primary sequence preference of casein kinases I and II, NIMA, phosphorylase kinase, calmodulin-dependent kinase II, CDK5 and Erkl. Mol. Cell. Biol. 16, 6486-6493.

van Leeuwen, J. E. M., and Samelson, L. E. (1999). T cell antigen-receptor signal transduction. Curr. Opin. Immunol. 11, 242-248.

Williams, E. J., Dunican, D. J., Green, P. J., Howell, F. V., Derossi, D., Walsh, F. S., and Doherty, P. (1997). Selective inhibition of growth factor-stimulated mitogenesis by a cell-permeable Grb2-binding peptide. J. Biol. Chem. 272, 22349-22354.

Zhao, Q., and Weiss, A. (1996). Enhancement of lymphocyte responsiveness by a gain-of-function mutation of ZAP-70. Mol. Cell. Biol. 16, 6765-6774.

U.S. Pat. No. 5,532,167

U.S. Pat. No. 6,004,757

PCT publication WO98/54577

Equivalents

Those skilled in the art will recognize, or be able to ascertain using no more than routine experimentation, many equivalents to the specific embodiments of the invention described herein. Such equivalents are intended to be encompassed by the following claims.

All references disclosed herein are incorporated by reference in their entirety.

What is claimed is:

1. A method for determining an amino acid sequence binding motif for a phosphorylation site of a kinase, comprising:
a) contacting the kinase with a peptide library, wherein each peptide comprises a single non-degenerate phosphorylatable amino acid in a fixed position of the peptide and wherein each peptide comprises one or more degenerate amino acids, under conditions which allow for binding of a peptide by the kinase at the phosphorylation site of the kinase;
b) allowing the kinase to bind peptides of the peptide library having a binding site for the kinase phosphorylation site to form kinase-peptide complexes;
c) isolating the kinase-peptide complexes from the unbound peptides;
d) releasing the peptides from the kinase-peptide complexes;
e) isolating the peptides previously bound to the kinase in kinase-peptide complexes;
f) determining the amino acid sequences of the peptides isolated in (e); and
g) determining an amino acid sequence motif for a binding site of the kinase based upon the relative abundance of different amino acid residues at each degenerate position within the peptides.

2. The method of claim 1, wherein the peptide library comprises peptides comprising the formula:
(Xaa)ₙ-Zaa-(Xaa)ₘ (SEQ ID NO: 13)
wherein Zaa is the single non-degenerate phosphorylatable amino acid and is selected from the group consisting of Tyr, Ser and Thr,
wherein Xaa is any amino acid except Zaa, and
wherein n and m are integers from 1-10 inclusive.

3. The method of claim 1, wherein the peptide library is a soluble synthetic peptide library.

4. The method of claim 1, wherein the single non-degenerate phosphorylatable amino acid is tyrosine.

5. The method of claim 4, wherein the peptides comprise the amino acid sequence Xaaₙ-Tyr-Xaaₘ (SEQ ID NO: 14), wherein Xaa is any amino acid except Tyr and wherein n and m are integers from 1-10 inclusive.

6. The method of claim 4, wherein the peptides comprise the amino acid sequence Xaaₙ-Tyr-Xaaₘ (SEQ ID NO: 15), wherein Xaa is any amino acid except Tyr or Cys and wherein n and m are integers from 1-10 inclusive.

7. The method of claim 4, wherein the peptides comprise the amino acid sequence Xaaₙ-Tyr-Xaaₘ (SEQ ID NO: 16), wherein Xaa is any amino acid except Tyr, Cys or Trp and wherein n and m are integers from 1-10 inclusive.

8. The method of claim 7, wherein the peptides comprise the amino acid sequence Xaa₄-Tyr-Xaa₄ (SEQ ID NO: 17), wherein Xaa is any amino acid except Tyr, Cys or Trp.

9. The method of claim 1, wherein the single non-degenerate phosphorylatable amino acid is serine.

10. The method of claim 9, wherein the peptides comprise the amino acid sequence Xaaₙ-Ser-Xaaₘ (SEQ ID NO: 18), wherein Xaa is any amino acid except Ser and wherein n and m are integers from 1-10 inclusive.

11. The method of claim 9, wherein the peptides comprise the amino acid sequence Xaaₙ-Ser-Xaaₘ (SEQ ID NO: 19), wherein Xaa is any amino acid except Ser or Cys and wherein n and m are integers from 1-10 inclusive.

12. The method of claim 9, wherein the peptides comprise the amino acid sequence Xaaₙ-Ser-Xaaₘ (SEQ ID NO: 20), wherein Xaa is any amino acid except Ser, Cys or Trp and wherein n and m are integers from 1-10 inclusive.

13. The method of claim 1, wherein the single non-degenerate phosphorylatable amino acid is threonine.

14. The method of claim 13, wherein the peptides comprise the amino acid sequence Xaaₙ-Thr-Xaaₘ (SEQ ID NO: 21), wherein Xaa is any amino acid except Thr and wherein n and m are integers from 1-10 inclusive.

15. The method of claim 13, wherein the peptides comprise the amino acid sequence Xaaₙ-Thr-Xaaₘ (SEQ ID NO: 22), wherein Xaa is any amino acid except Thr or Cys and wherein n and m are integers from 1-10 inclusive.

16. The method of claim 13, wherein the peptides comprise the amino acid sequence Xaaₙ-Thr-Xaaₘ (SEQ ID NO: 23), wherein Xaa is any amino acid except Thr, Cys or Trp and wherein n and m are integers from 1-10 inclusive.

17. The method of claim 1, wherein the peptide library is contacted with the kinase by application of the library to a substrate to which the kinase is immobilized.

18. The method of claim 1, wherein the kinase-peptide complexes are isolated by washing the kinase-peptide complexes in a buffer that permits binding of peptides to the phosphorylation site of the kinase.

19. The method of claim 1, wherein the peptides are eluted from the kinase-peptide complexes by incubating the kinase-peptide complexes with an elution solution.

20. The method of claim 19, wherein the elution solution has an acidic pH.

21. A kinase binding molecule comprising a binding motif for a phosphorylation site of a kinase identified according to any of claims 1-20.

22. The kinase binding molecule of claim 21, further comprising a molecule that mediates transport across a plasma membrane.

23. The kinase binding molecule of claim 22, wherein the molecule that mediates transport across a plasma membrane is selected from the group consisting of penetratin, Tat, VP22, Pep-1 and fragments thereof that mediate transport across a plasma membrane.

24. A composition comprising the kinase binding molecule of claim 21 and a pharmaceutically acceptable carrier.

25. Use of the kinase binding molecule of claim 21 in the preparation of a medicament.

26. A method for inhibiting phosphorylation of proteins by a kinase, comprising,
contacting the kinase with an amount of the kinase binding molecule of any of claims 21-23 or the composition of claim 24 effective to inhibit the phosphorylation.

27. A method for treating a condition that includes phosphorylation of proteins by a kinase, comprising,
administering to a subject an amount of the kinase binding molecule of any of claims 21-23 or the composition of claim 24 effective to inhibit the phosphorylation of the proteins by the kinase.

28. The method of claim 27, wherein the subject is a mammal.

29. The method of claim 27, wherein the subject is a human.

30. A kinase inhibitor comprising a binding motif for a phosphorylation site of a kinase identified according to any of claims 1-20, wherein the single non-degenerate phosphorylatable amino acid is replaced by an amino acid that cannot be phosphorylated by the kinase to which the inhibitor binds.

31. The kinase inhibitor of claim 30, wherein the binding motif comprises SEQ ID NO: 2.

32. The kinase inhibitor of claim 30, wherein the binding motif comprises SEQ ID NO: 4.

33. The kinase inhibitor of claim 31 or 32, wherein the amino acid that cannot be phosphorylated is Tyr and the Tyr is replaced by Phe or a halogenated Phe.

34. The kinase inhibitor of any of claims 30-33, further comprising a molecule that mediates transport across a plasma membrane.

35. The kinase inhibitor of claim 34, wherein the molecule that mediates transport across a plasma membrane is selected from the group consisting of penetratin, Tat, VP22, Pep-1 and fragments thereof that mediate transport across a plasma membrane.

36. A composition comprising the kinase inhibitor of any of claims 30-33 and a pharmaceutically acceptable carrier.

37. Use of the kinase inhibitor of claim 30 in the preparation of a medicament.

38. A method for inhibiting phosphorylation of proteins by a kinase, comprising,
contacting the kinase with an amount of the kinase inhibitor of any of claims 30-35 or the composition of claim 36 effective to inhibit the phosphorylation.

39. A method for treating a condition that includes phosphorylation of proteins by a kinase, comprising,
administering to a subject an amount of the kinase inhibitor of any of claims 30-35 or the composition of claim 36 effective to inhibit the phosphorylation of the proteins by the kinase.

40. The method of claim 39, wherein the subject is a mammal.

41. The method of claim 40, wherein the subject is a human.

42. A method for validating a kinase as a target for inhibition for the treatment of a condition, comprising
providing a molecule comprising a binding motif for a phosphorylation site of a kinase as claimed in any of claims 1-20,
contacting a biological sample containing a kinase suspected of being involved in the causation of the condition with the molecule for a time sufficient to permit binding of the molecule and the kinase, and
determining the effect of the molecule on one or more biological processes mediated by the kinase.

43. The method of claim 42, wherein the biological sample is a cell.

44. The method of claim 42, wherein the biological sample is an organism.

45. The method of claim 43 or 44, wherein the cell or organism is a model system for the condition.

46. A method for inhibiting a ZAP-70 kinase comprising,
contacting the ZAP-70 kinase with an amount of a kinase inhibitor as claimed in any of claims 30-35, effective to inhibit the ZAP-70 kinase.

47. The method of claim 46, wherein the kinase inhibitor comprises SEQ ID NO: 4 or SEQ ID NO: 9.

48. A method for treating a condition mediated by a ZAP-70 kinase comprising
administering to a subject in need of such treatment an amount of a kinase inhibitor as claimed in any of claims 30-35 or a composition as claimed in claim 36, effective to inhibit the ZAP-70 kinase.

49. The method of claim 48, wherein the kinase inhibitor comprises SEQ ID NO: 4 or SEQ ID NO: 9.

50. The method of claim 49, wherein the subject is a mammal.

51. The method of claim 50, wherein the subject is a human.

52. A method for inhibiting transcription mediated by a ZAP-70-responsive promoter sequence, comprising
contacting a biological sample, cell or organism that comprises a ZAP-70-responsive promoter sequence operably linked to a nucleic acid molecule with an amount of a kinase inhibitor as claimed in any of claims 30-35 effective to inhibit the transcription of the nucleic acid molecule mediated by the ZAP-70-responsive promoter sequence.

53. The method of claim 52, wherein the kinase inhibitor comprises SEQ ID NO: 4 or SEQ ID NO: 9.

54. The method of claim 52, wherein the ZAP-70-responsive promoter sequence is an interleukin-2 promoter.

55. A method for treating a condition mediated by transcription mediated by a ZAP-70-responsive promoter sequence, comprising
administering to a subject in need of such treatment an amount of a kinase inhibitor as claimed in any of claims 30-35 or a composition as claimed in claim 36, effective to inhibit the transcription mediated by the ZAP-70-responsive promoter sequence.

56. The method of claim 55, wherein the kinase inhibitor comprises SEQ ID NO: 4 or SEQ ID NO: 9.

57. The method of claim 55, wherein the ZAP-70-responsive promoter sequence is an interleukin-2 promoter.

58. The method of claim 55, wherein the subject is a mammal.

59. The method of claim 58, wherein the subject is a human.

60. A method for identifying a kinase inhibitor compound, comprising
providing a kinase, a kinase inhibitor that binds the kinase, and a candidate kinase inhibitor compound,
contacting the kinase with the candidate kinase inhibitor compound and the kinase inhibitor under conditions that permit binding of the kinase inhibitor to the kinase, wherein either or both of the candidate kinase inhibitor compound and the kinase inhibitor are detectable, and wherein either or both of the candidate kinase inhibitor compound and the kinase inhibitor comprises a sequence determined according to any of claims 1-20,
separating the kinase from the unbound kinase inhibitor and unbound candidate kinase inhibitor compound, and
detecting the amounts of detectable kinase inhibitor and/or the detectable candidate kinase inhibitor compound bound to the kinase as a measure of the presence of a candidate kinase inhibitor compound that competes with the kinase inhibitor for binding to the kinase.

61. The method of claim 60, further comprising testing the activity of the kinase in the presence of the candidate kinase inhibitor compound, wherein a greater reduction in kinase activity in the presence of the candidate kinase inhibitor compound than in the absence of the candidate kinase inhibitor compound indicates that the candidate kinase inhibitor compound is a kinase inhibitor.

62. The method of claim 60, wherein the candidate kinase inhibitor compound or the kinase inhibitor comprises a binding motif sequence comprising SEQ ID NO: 13.

63. The method of claim 60, wherein the candidate kinase inhibitor compound is a small organic molecule.

64. A kinase inhibitor compound identified according to any one of claims 60-63.

65. Use of the kinase inhibitor compound of claim 64 in the preparation of a medicament.


SEQUENCE LISTING (23 sequences)

SEQ ID NO: 1 - protein, 15 aa, Artificial Sequence
  Synthetic Peptide
MAXXXXYXXXXAKKK

SEQ ID NO: 2 - protein, 9 aa, Artificial Sequence
  Synthetic Peptide
XXXXYXXXX

SEQ ID NO: 3 - protein, 9 aa, Artificial Sequence
  Synthetic Peptide
EEEEYFFII

SEQ ID NO: 4 - protein, 9 aa, Artificial Sequence
  Synthetic Peptide
KLILYLLLL

SEQ ID NO: 5 - protein, 13 aa, Artificial Sequence
  Synthetic Peptide
EEEEYFFIIAKKK

SEQ ID NO: 6 - protein, 13 aa, Artificial Sequence
  Synthetic Peptide
KLILYLLLLAKKK

SEQ ID NO: 7 - protein, 4 aa, Artificial Sequence
  Synthetic Peptide
AKKK

SEQ ID NO: 8 - protein, 13 aa, Artificial Sequence
  Synthetic Peptide
ELILYLLLLAKKK

SEQ ID NO: 9 - protein, 9 aa, Artificial Sequence
  Synthetic Peptide
KLILFLLLL

SEQ ID NO: 10 - protein, 8 aa, Artificial Sequence
  Synthetic Peptide
LILYLLLL

SEQ ID NO: 11 - protein, 16 aa, Artificial Sequence
  Synthetic Peptide
RQIKIWFQNRRMKWKK

SEQ ID NO: 12 - protein, 25 aa, Artificial Sequence
  Synthetic Peptide
RQIKIWFQNRRMKWKKKLILFLLLL

SEQ ID NO: 13 - protein, 21 aa, Artificial Sequence
  Synthetic Peptide
XXXXXXXXXXXXXXXXXXXXX

SEQ ID NO: 14 - protein, 21 aa, Artificial Sequence
  Synthetic Peptide
XXXXXXXXXXYXXXXXXXXXX

SEQ ID NO: 15 - protein, 21 aa, Artificial Sequence
  Synthetic Peptide
XXXXXXXXXXYXXXXXXXXXX

SEQ ID NO: 16 - protein, 21 aa, Artificial Sequence
  Synthetic Peptide
XXXXXXXXXXYXXXXXXXXXX

SEQ ID NO: 17 - protein, 9 aa, Artificial Sequence
  Synthetic Peptide
XXXXYXXXX

SEQ ID NO: 18 - protein, 21 aa, Artificial Sequence
  Synthetic Peptide
XXXXXXXXXXSXXXXXXXXXX

SEQ ID NO: 19 - protein, 21 aa, Artificial Sequence
  Synthetic Peptide
XXXXXXXXXXSXXXXXXXXXX

SEQ ID NO: 20 - protein, 21 aa, Artificial Sequence
  Synthetic Peptide
XXXXXXXXXXSXXXXXXXXXX

SEQ ID NO: 21 - protein, 21 aa, Artificial Sequence
  Synthetic Peptide
XXXXXXXXXXTXXXXXXXXXX

SEQ ID NO: 22 - protein, 21 aa, Artificial Sequence
  Synthetic Peptide
XXXXXXXXXXTXXXXXXXXXX

SEQ ID NO: 23 - protein, 21 aa, Artificial Sequence
  Synthetic Peptide
XXXXXXXXXXTXXXXXXXXXX